(19) United States
(12) Patent Application Publication
(10) Pub. No.: US 2009/0299096 A1
(43) Pub. Date: Dec. 3, 2009

(54) Amidoadamantanes and Method for Producing the Same

(76) Inventors: Peter R. Schreiner, Wettenberg (DE); Andrey A. Fokin, Giessen (DE); Lukas Wanka, Wollstadt (DE); Derek M. Wolfe, Athens, GA (US)

Correspondence Address: CLARK & BRODY, 1090 VERMONT AVENUE, NW, SUITE 250, WASHINGTON, DC, 20005, US

(21) Appl. No.: 12/224,545

(22) PCT Filed: Feb. 20, 2007
(86) PCT No.: PCT/EP2007/001431

§ 371 Date: Jan. 21, 2009

(30) Foreign Application Priority Data
Mar. 1, 2006 (DE) 10 2006-009 278.3

(51) Int. Cl. C07C 233/03; C07C 231/08

(52) U.S. Cl. 564/217

(57) ABSTRACT

The invention relates to 1-formamido-adamantane derivatives of formula (I), which carry the substituents R¹, R² or R³ in 3, 5 and/or 7 position, the substituents being defined as follows: hydrogen, a linear or branched alkyl, alkenyl or alkinyl group with up to 6 C atoms, an aliphatic or aromatic, cyclic or heterocyclic hydrocarbon group with up to 10 carbon atoms, and formamides of hydrocarbons from the group of diamondoids, for example diamantane, triamantane, tetramantane and pentamantane, however, with the exception of 1-formamido-3,5-dimethyladamantane. The invention also relates to a method for the direct formation of formamide or acetamide of adamantane, adamantane derivatives with the aforementioned definition of substituents or of a hydrocarbon from the group of diamondoids, characterized by reacting the adamantane, the adamantane derivative or the diamondoid with formamide, acetamide or acetonitrile in concentrated acids, while avoiding SO₃ containing sulfuric acid or 100% azotic acid.

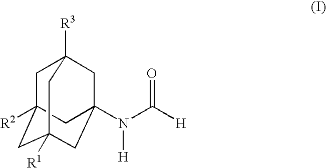

Subject matter of the invention are 1-formamidoadamantane derivatives and a method for the direct formamide or acetamide formation of adamantane or adamantane derivatives. 1-formamido-3,5-dimethyladamantane and methods for producing the same are not the subject matter of the present invention. 1-formamido- and 1-acetamidoadamantane derivatives are important intermediates in the production of aminoadamantanes, wherein a tertiary H-atom of the adamantane skeleton is substituted by an amino group. Aminoadamantanes are defined by their interesting biological effects. The 1-amino-3,5-dimethyladamantane has already found application as a pharmaceutical for the treatment of the Alzheimer's disease under the name of the active agent memantine. Therefore, there is great interest to create simple synthesis possibilities for this compound and other aminoadamantanes in order to examine the effects of further representatives of this compound class. As, according to present knowledge, aminoadamantanes are preferably accessible through acid hydrolysis of corresponding amidoadamantanes, the aim is thus to develop new amidoadamantanes and methods for producing the same.

Until now, the synthesis of amidoadamantanes has taken a synthesis pathway in which halogenated adamantanes were used as starting materials. The halogenated adamantane was then reacted with an acid amide, e.g. acetamide, by cleavage of hydrogen halide. This method, however, has the disadvantage that halogen-containing by-products are formed, whose disposal is laborious and costly.

The aim was therefore to find a method for the direct amide formation of adamantanes. The term “direct” amide formation is understood as the introduction of an amide group, e.g. a formamide group or acetamide group, into the adamantane molecule, without a prior halogenation.

The synthesis of acetamides of cyclic and polycyclic alkanes is a method known in literature. Usually one proceeds from brominated precursors, which are then reacted with a nitrile or hydrogen cyanide under strongly acid conditions in a Ritter-type reaction.

Several methods are known for the introduction of formamide and/or acetoamido groups. Haaf (Angew. Chem. 1961, 73, 144) produced 1-formamidoadamantane by reacting adamantane with t.-butyl cations under strongly acid conditions, with the use of hydrogen cyanide as nucleophile. In later studies by Haaf (Chem. Ber. 1963, 96(12), 3359-3369), the substitution of tertiary carboxylic acid groups or ester groups by amides with hydrogen cyanide or nitriles in conc. H₂SO₄ has been described. As, for example, through the reaction of adamantane-1-carboxylic acid with 100% sulfuric acid and NaCN, 1-formamidoadamantane with 56% yield was obtained; through the reaction with acetonitrile, 1-acetamidoadamantane was obtained. Gerzon et al. (J. Med. Chem. 1963, 6, (6), 760-763) produced acetamides of the adamantane and some derivates from brominated precursors through reaction with nitriles in conc. sulfuric acid. In Gerzon, K.; Tobias, D. J.; Holmes, R. E.; Rathbun, R. E.; Kattau, R. W., J. Med. Chem. 1967, 10, (4), 603-606, formamides of the adamantane were produced from the brominated precursors. N-acyl-1-amino-3,5,7-trimethyladamantanes were produced here through the reaction of 1-bromo-3,5,7-trimethyladamantane with amides, for example formamide, at 125-130° C. In BE 648 581 196 40 703 1-formamidoadamantane, as well as 1-acetamidoadamantane were produced by adding dropwise conc. sulfuric acid to a mixture of 1-bromoadamantane and the corresponding nitrile or hydrogen cyanide. In NL 6 604 975 196 61 024, furthermore, the aminoadamantanes were reacted with Ag₂SO₄ and N-methylamides in the excess into N-methylase adamantylamides. In NL 6 604 975 196 61 024 aminoadamantanes were produced through amide formation of the brominated or hydroxylated precursors with hydrogen cyanide or nitriles in 100-110% sulfuric acid. Skoldinov et al. (Khimiko-Farmatsevticheskii Zhumal 1967, 1(8), 22-26) produced diamino-diadamantyl compounds through a Ritter reaction of the brominated precursors in conc. sulfuric acid. Fonken et al. (J. Org. Chem. 1968, 33(8), 3201-3207) produced 1-formamidoadamantane from the formic acid salt of the 1-aminoadamantane through reaction with acetic anhydride. Aldrich et al. (J. Med. Chem. 1971, 14 (6), 535-543) produced 1-formamidoadamantane through reaction of 1-bromoadamantane with formamide in the presence of Ag₂SO₄. Jones und Mellor (Synthesis 1976, (1), 32-33) synthesized formamides and acetamides from, amongst others, adamantane and 1,3-dimethyladamantane through a lead tetraacetate induced Ritter reaction with NaCN (the formamides production) and acetonitrile (the acetamides production). Druelinger et al. (J. Heterocycl. Chem. 1976, 13(5), 1001-1007) produced 1-formamidoadamantane through photochemical reaction from N-adamantyl oxaziridine. In SU 408 546 197 61 205 a variant of the production of 1-aminoadamantane from 1-formamidoadamantane has been described, which in turn was produced from 1-chloroadamantane or 1-bromoadamantane with an excess of formamide at high temperatures. Olah et al. (Synthesis 1979, (4), 274-276) produced acetamidoadamantanes by reacting halogenated adamantanes with NOBF₄ in acetonitrile. Sasaki et al. (J. Org. Chem. 1981, 46(26), 5445-5447) produced 1-formamidoadamantane from 1-bromadamantane through reaction with trimethylsilylcyanide, under TiCl₄ catalysis EP 89-106 657 198 90 414 describes the preparation of a series of differently substituted 1-acetamidoadamantane and/or 1-formamidoadamantane derivatives from 1-bromoadamantanes as precursors; here, for example, 1-bromo-3-phenyladamantane with an excess of formamide was heated for a considerable time, wherein 1-N-formamido-3-phenyladamantane was formed with 80% yield.

Barton et al. (Tetrahedron 1993, 49(33), 7193-7214) produced 1-formamidoadamantane by heating 1-aminoadamantane and ethyl formiate in DMSO. Goel et al. (Tetrahedron Lett. 1996, 37(45), 8129-8132) describe an extension of the Ritter reaction for the preparation of formamides from alcohols, amongst others adamantane-1-ol, using trimethylsilylcyanide. Kovalev et al. (Synthesis 1997, (9), 1034-1040) have described the preparation of 1-acylaminoadamantanes from adamantane-1-ol, with nitriles, ureas or carboxylic acid amides in hot trifluoroacetic acid. In the Russian patent application RU 98-116 071 199 80 824 various 1-aminoadamantane derivatives, 1-formamidoadamantane amongst others, synthesized through the reaction of 1,3-dehydroadamantane with excess amine (partially in the presence of bortrifluoride etherate as catalysts) were disclosed.

The most well-known methods by far for the amide formation of branched hydrocarbons proceed from halogen alkanes or the corresponding alcohols. An additional synthesis step is required for their preparation, often with a purification of the intermediates. Usually large excesses of elementary bromine are used in the bromination of hydrocarbons, which makes the reactions expensive, dangerous and, with regard to the disposal of the bromine waste, unattractive. The most methods known in literature for the preparation of adamantane formamides and its derivatives use the very poisonous hydrogen cyanide. The methods known in the literature for the amide formation of alcohols partly use dangerous (trimethylsilylcyanide) and expensive reagents and solvents (trifluoroacetic acid).

The known bromine-free methods use organic co-solvents, which lead to impurities in the products, or use the poisonous environmentally dangerous and reproduction toxic lead tetraacetate.

Finally, the production of stabilomers such as adamantane or 1,3-dimethyladamantane takes place commercially by using large amounts of metal-containing Lewis acids, such as anhydrous AlCl₃, which leads to a considerable amount of waste generation.

A method for the direct and halogen-free amide formation of adamantane derivatives, such as adamantane-1-carboxylic acids or 1,3-dimethyladamantane using inexpensive reagents is described in the WO 2006/010362. The direct synthesis of the adamantane formamides, the 1,3-dimethyladamantane or other adamantine derivatives without prior reactions to activated adamantane derivatives (such as to the alcohols or the bromine compounds) is not described in the literature.

An in-situ synthesis of the hydrocarbon to be directly amide-formated, such as adamantane or 1,3-dimethyladamantane, under acid conditions with subsequent amide formation to formamide or to other acylamines is not described in the literature.

It is the aim of the invention to provide simplified methods for the selective introduction of amino groups in adamantane und its derivatives. In doing so, the prior halogenation of the starting materials is to be avoided. Furthermore, the methodology described here comprises a favorable impurity profile, as it proceeds with high yields and with a low ratio of by-products, tolerating some impurity of the starting materials. An in-situ production of the starting material with inexpensive precursors under the same conditions (one pot method) is made possible. The bromine-free synthesis of more suitable, for the subsequent synthesis operations, is to be enabled as well.

It has now been found that this aim is achieved by the new 1-formamidoadamantane of the formula I,

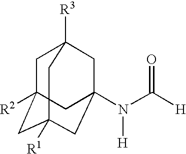

which carry in positions 3, 5 and/or 7 the substitutes R¹, R² or R³, being defined as follows:
hydrogen, a linear or branched alkyl, alkenyl or alkinyl group with up to 10 C-atoms,
an aliphatic or aromatic, cyclic or heterocyclic hydrocarbon residue with up to 10 carbon atoms,
wherein, however, the 1-formamido-3,5-dimethyladamantane and methods for its production are excluded.

Furthermore, it was found that the formamides and acylamides of other hydrocarbons in the diamondoid series, for example diamantane, triamantane, tetramantane and pentamantane, achieve this aim.

These 1-formamidoadamantanes, but also the in principle already known acetamidoadamantanes, are produced by a direct amide formation, in which the adamantane or the adamantane derivative is reacted with formamide, acetamide or acetonitrile in concentrated acids, wherein the direct formamide formation of 1,3-dimethyladamantane is excluded.

30 to 70%, in particular 65% nitric acid and 90 to 100%, above all 95 to 98% sulfuric acid are preferably used as concentrated acids. However, also 85 to 100% phosphoric acid, perchloric acid, disulfuric acid or chlorosulfuric acid are suitable to be used. The reaction generally takes place at −40° C. to 50° C., preferably at 0° C. Yields of 40 to 95% are usually achieved in the method according to the present invention.

This method is not only distinguished by the absence of halogens, but also comprises a more favorable impurity profile, as it proceeds with high yields and with only a low ratio of by-products, even tolerating some impurity of the starting materials. Even an in-situ production of the starting materials from inexpensive precursors is possible, so that the desired end product is available in a one pot method. Thereby e.g. the dialkyladamantane to be reacted in situ from a perhydrogenated alkylcyclopentadiene dimer, produced through cationic rearrangement and then, the amide formation of the resulting diakyladamantane is suitable for being conducted in the same reaction vessel under acid conditions.

The treatment of the perhydrogenated alkylcyclopentadiene dimer with concentrated acids allows the synthesis of the starting material diakyladamantane in situ, before the amide formation according to the following reaction:

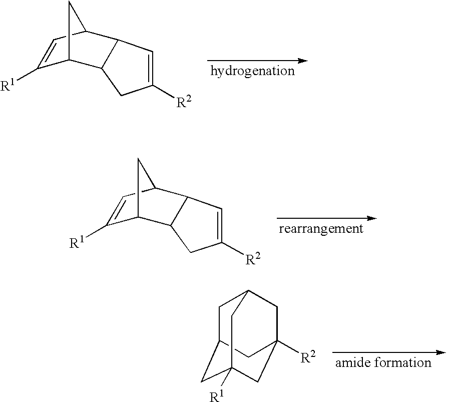

The method described in the international application PCT/DE2005/001304 is based on the production of carbocations in concentrated acids, which are quenched by a nucleophile, for example a nitrile, and through an aqueous workup convert into the corresponding 1-amidoadamantane derivatives according to the following reaction:

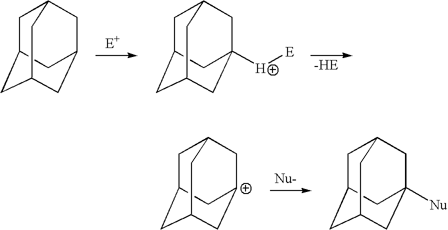

The optimization of the reaction conditions in the synthesis of 1-formamidoadamantane creates the possibility of conducting a milder reaction process under complete avoidance of the use of oleum or 100% nitric acid.

The invention is described in more detail through the following embodiment:

Direct Amide Formation of 1-methyladamantane with Formamide

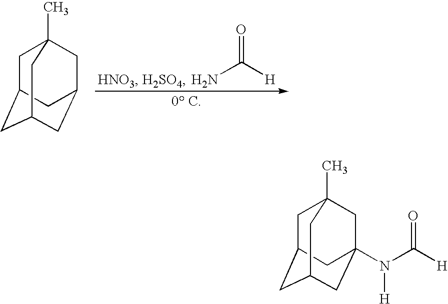

1 mL technical grade nitric acid (65%) is added to 1.5026 g (10 mmol) 1-methyladamantane, while cooling to 0° C. in a heated 100 mL round bottom flask with an active magnetic stirrer. Subsequently, 25 mL technical grade sulfuric acid (95-98%) are added dropwise at 0° C. within 3 hours. One stirs for 12 h at 0° C. and further 10 mL 95-98% sulfuric acid are added. After 3 further hours of stirring at 0° C., the resulting solution is added dropwise to 50 mL technical grade formamide, within one hour of intensive stirring under exclusion of moisture at 0° C. This mixture is then stirred again for 30 min at 0° C. and 120 min at room temperature, after which at first 100 mL dichloromethane are added. The phases are separated, the aqueous phase is extracted with dichloromethane (2×20 mL), and the combined organic phases are dried over Na₂SO₄. After the desiccant has been filtered off, the solvents are largely removed from the solution by rotary evaporation. The remaining yellowish crude product is purified by column chromatography (170 g SiO₂, acetic acid ethyl ester, Rf=0.37). After careful evaporation of the eluent, one obtains 0.864 g (45%) 1-formamido-3-methyladamantane as weakly yellowish, viscose oil, crystallizing into colorless needles during prolonged standing.

¹H NMR (200 MHz, CDCl₃): δ/ppm=8.25 (d, J=12.4 Hz) and 8.05 (d, J=9.0 Hz), together 1H, NHCHO (2 isomers); 6.62 (bs) and 5.33 (bs), together 1H, NHCHO (2 isomers); 2.22-1.33 (m, 14H, CH/CH₂ of the adamantane); 0.87 (s) and 0.85 (s), together 3H, —CH₃ (2 isomers).

¹³C NMR (50 MHz, CDCl₃): δ/ppm=162.6 and 160.4 (C═O), 53.0 and 51.6, 50.7 and 48.4, 43.3 and 43.2, 42.9 and 41.1, 35.5 and 35.1, 32.0 and 31.9, 30.4 and 30.3, 29.7 and 29.6 (signals doubled, as there are two isomers).

IR (KBr): \tilde{ν}/cm⁻¹=3439, 3211, 3083, 2902, 2849, 1696, 1675, 1536, 1456, 1313.

MS: m/z=193 (M⁺), 178, 148, 136, 122, 106, 92, 79

1. 1-formamidoadamantane derivative of the formula I 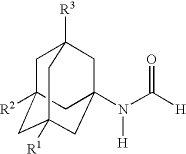
which carries in 3, 5 and/or 7 position the substituents R¹, R² or R³, being defined as follows:
hydrogen, a linear or branched alkyl, alkenyl or alkinyl group with up to 6 C atoms, an aliphatic or aromatic, cyclic or heterocyclic hydrocarbon residue with up to 10 carbon atoms, wherein, the adamantane ring system is able to be replaced by a hydrocarbon from the diamondoid series, however the 1-formamido-3,5-dimethyladamantane is excluded.

2. Method for the formamide or acetamide formation of adamantane or an adamantane derivative of the formula II 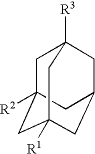
which carries in 3, 5 and/or 7 position the substituents R¹, R² or R³, being defined as follows:
hydrogen, a linear or branched alkyl, alkenyl or alkinyl group with up to 6 C atoms, an aliphatic or aromatic, cyclic or heterocyclic hydrocarbon residue with up to 10 carbon atoms, or from a hydrogen carbon from the diamondoid series wherein, the adamantane, the adamantane derivative or the diamondoid is reacted with formamide, acetamide or acetonitrile in concentrated acids, but avoiding SO₃-containing sulfuric acid or 100% nitric acid, wherein the direct formamide formation of 1,3-dimethyladamantane is excluded.

3. Method according to claim 2, wherein, a dialkyladamantane is used as adamantane derivative.

4. Method according to claim 2, wherein, the adamantane or the adamantane derivative is produced before the direct formamide or acetamide formation, from a cyclopentadiene dimer or one of its derivatives through perhydrogenation in a separate reaction or in situ.

5. Method according to claim 2, wherein, the formamidoadamantane or acetamidoadamantane obtained is converted into the corresponding aminoadamantane by hydrolysis.

6. Method according to claim 3, wherein, the adamantane or the adamantane derivative is produced before the direct formamide or acetamide formation, from a cyclopentadiene dimer or one of its derivatives through perhydrogenation in a separate reaction or in situ.

7. Method according to claim 3, wherein, the formamidoadamantane or acetamidoadamantane obtained is converted into the corresponding aminoadamantane by hydrolysis.

8. Method according to claim 4, wherein, the formamidoadamantane or acetamidoadamantane obtained is converted into the corresponding aminoadamantane by hydrolysis.
